(19) United States
(12) Patent Application Publication
(10) Pub. No.: US 2026/0097218 A1
(43) Pub. Date: Apr. 9, 2026

(54) PRE-SHOCK COMMUNICATION WINDOW TO SYNCHRONIZE COORDINATED THERAPY

(71) Applicant: Stryker Corporation, Portage, MI (US)

(72) Inventors: David J. Linville, Redmond, WA (US); Tyson G. Taylor, Bothell, WA (US); Rose Tingwei Yin, Bellevue, WA (US); Fred W. Chapman, Newcastle, WA (US); Robert G. Walker, Seattle, WA (US); Ryan William Apperson, Bothell, WA (US); Rockland W. Nordess, Kirkland, WA (US); Reza Sharif, Lake Forest, WA (US)

(73) Assignee: Stryker Corporation, Portage, MI (US)

(21) Appl. No.: 19/352,250

(22) Filed: Oct. 7, 2025

Related U.S. Application Data
(60) Provisional application No. 63/705,457, filed on Oct. 9, 2024.

(51) Int. Cl. A61N 1/39

(52) U.S. Cl. CPC A61N 1/3904; A61N 1/3912; A61N 1/3918; A61N 1/3925; A61N 1/3987; A61N 1/3993

(57) ABSTRACT

An example method includes receiving, by a defibrillator operating in a multi-shock mode, an input signal associated with a multi-shock therapy. The multi-shock therapy includes a first shock administered to a subject and a second shock administered to the subject. The first shock and the second shock are temporally overlapping. In response to expiration of a predetermined delay period after receiving the input signal, the defibrillator outputs the first shock to the subject.

### CROSS-REFERENCE TO RELATED APPLICATION

This application claims the priority of U.S. Provisional App. No. 63/705,457, which was filed on Oct. 9, 2024 and is incorporated by reference herein in its entirety.

### BACKGROUND

Particular heart arrhythmias, such as ventricular fibrillation (VF) and pulseless ventricular tachycardia (VT), are deadly if untreated. An individual with one of these arrhythmias can be treated by administering an electrical shock to the individual's heart. This treatment is known as defibrillation. The arrhythmias that are treatable by defibrillation are known as shockable arrhythmias.

Unfortunately, many instances of shockable arrhythmias are resistant to conventional defibrillation therapies. For instance, an individual is determined to have refractory VF if they have VF that continues through the administration of an electrical shock. Recently, double sequential defibrillation (DSD) (also referred to as “double sequential external defibrillation” or “DSED”) has been proposed as an alternative to single-shock defibrillation. A DSD therapy, for instance, can be administered by discharging two electrical shocks to an individual's heart, rather than one. Researchers have suggested that DSD administration can increase survivability of refractory VF. Cheskes et al., 387 N. Engl. J. Med. 1947 (2022).

### BRIEF DESCRIPTION OF THE DRAWINGS

FIG. 1 illustrates an environment in which a subject is treated with a multi-shock therapy (e.g., a double-sequential defibrillation (DSD) therapy) by two separate medical devices.

FIG. 2 illustrates an example timing relationship between actions of a primary defibrillator and actions of a secondary defibrillator configured to administer a multi-shock therapy.

FIG. 3 illustrates an example process for coordinating a multi-shock therapy using a delay period.

FIG. 4 illustrates an example process for protecting circuitry associated with a defibrillator during administration of a multi-shock therapy

FIG. 5 illustrates an example of an external defibrillator configured to perform various functions described herein.

FIGS. 6A and 6B illustrate examples of environments and timing related to administering a multi-shock therapy. FIG. 6A shows an environment configured to administer the multi-shock therapy. FIG. 6B shows a timing relationship of multiple shocks administered in the multi-shock therapy.

### DETAILED DESCRIPTION

Various implementations described herein relate to systems, devices, and methods for administration of multi-shock therapies, such as double-sequential defibrillation (DSD) therapies. Previously, a user could administer a multi-shock therapy to an individual by manually activating two separate defibrillators connected to the individual. However, manually activating two separate defibrillators has some drawbacks. Namely, certain timing relationships between electrical shocks may reduce the efficacy of the multi-shock therapy, or even cause the multi-shock therapy to harm the individual being treated. Moreover, it is possible that an electrical shock output by one defibrillator can harm sensitive circuitry of the other defibrillator during administration of the multi-shock therapy.

Implementations of the present disclosure address these and other problems by providing a mechanism for two separate defibrillators to discharge electrical shocks at a precise and predetermined timing relationship. In various cases, a primary defibrillator is configured to coordinate timing of a multi-shock therapy with a secondary defibrillator. For instance, a primary defibrillator operating in a multi-shock mode may output an instruction, to the secondary defibrillator, that causes the secondary defibrillator to output an electrical shock that has a precise timing relationship with respect to an electrical shock output by the primary defibrillator. In some cases, the instruction is output in response to the primary defibrillator receiving an input signal from a user. In various cases, to accommodate for potential delays and latency of communications between the primary defibrillator and the secondary defibrillator, both shocks are scheduled after expiration of a predetermined delay period, which may extend from the time that the primary defibrillator receives the input signal or the time that the primary defibrillator outputs the instruction to the secondary defibrillator. The delay period, for example, can increase the chance that both defibrillators are prepared to administer their respective electrical shocks at a preferred timing relationship.

Some implementations of the present disclosure also reduce the likelihood that one defibrillator will be damaged by the electrical shock output by the other defibrillator. In some cases, an instruction from the primary defibrillator causes the secondary defibrillator to activate a multi-shock mode. In the multi-shock mode, the secondary defibrillator may deactivate one or more sensors of the secondary defibrillator, in order to prevent the sensor(s) from being damaged by the electrical shock output by the primary defibrillator. In some cases, the multi-shock mode causes the secondary defibrillator to deactivate its display, speaker, or other output devices, to allow the user to focus on alarms output by the primary defibrillator rather than being distracted by alarms from both defibrillators. In some examples, the primary defibrillator is configured to protect itself from a shock output by the secondary defibrillator.

Implementations of the present disclosure will now be described with reference to the accompanying claims.

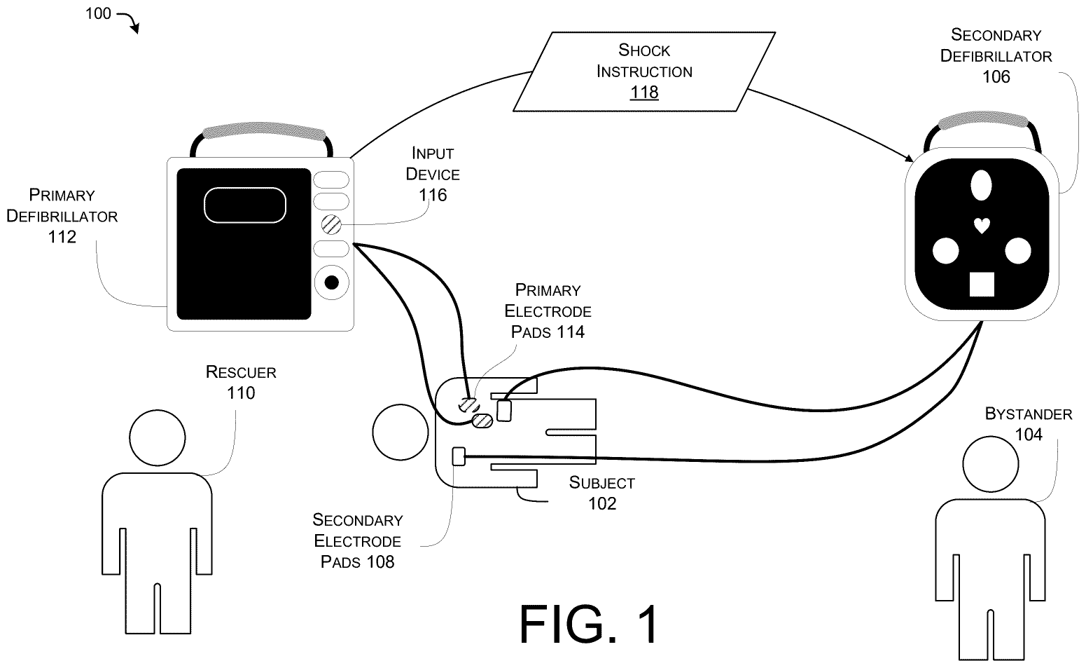

FIG. 1 illustrates an environment 100 in which a subject 102 is treated with a multi-shock therapy by two separate medical devices. In some cases, the environment 100 is a clinical environment, such as a hospital. In various cases, the environment 100 is in an out-of-hospital environment. For example, the subject 102 may have experienced a medical emergency in a non-hospital, public space, such as a library, airport terminal, school, or office building. In some cases, the subject 102 has collapsed or otherwise lost consciousness within the environment 100. In some examples, the subject 102 has experienced one or more other types of symptoms associated with a serious health condition. As a result, a bystander 104 may have contacted emergency services personnel in order to assist the subject 102 and to transport the subject 102 to a clinical environment, if necessary. In some cases, the bystander 104 is a healthcare professional, such as a nurse, emergency medical technician (EMT), paramedic, physician, physician's assistant, or other individual with specialized medical training.

Before the emergency services personnel have arrived to the environment 100, the bystander 104 has operated a secondary defibrillator 106 in order to monitor and potentially treat the subject 102. In some cases, the secondary defibrillator 106 is a monitor-defibrillator.

In various cases, the secondary defibrillator 106 is a public-access automated external defibrillator (AED). For instance, the secondary defibrillator 106 is designed for operation by the bystander 104, who does not have specialized medical knowledge.

In various cases, the secondary defibrillator 106 instructs the bystander 104 to apply secondary electrode pads 108 to the skin of the subject 102. For instance, the secondary electrode pads 108 may be adhered to skin on the chest of the subject 102. In various cases, under direction of the secondary defibrillator 106, the bystander 104 applies the secondary electrode pads 108 to the subject 102 along a first shock vector.

The secondary defibrillator 106 includes one or more measurement circuits configured to detect one or more physiological parameters of the subject 102. The measurement circuit(s), for instance, are configured to be connected to sensors that are applied to the body of the subject 102. The sensor(s), for instance, are configured to generate electrical signals that are indicative of the physiological parameter(s) of the subject 102. The measurement circuit(s) may infer the physiological parameter(s) based on the electrical signals generated by the sensor(s).

In particular cases, the secondary electrode pads 108 include electrodes that are configured to detect an electrical signal output by the heart of the subject 102 over time. A measurement circuit in the secondary defibrillator 106, for instance, is configured to detect an electrocardiogram (ECG) of the subject 102 based on the electrical signal detected by the electrodes.

In various implementations, the secondary defibrillator 106 is configured to identify a condition of the subject 102 based, at least in part, on the physiological parameter(s). For instance, the secondary defibrillator 106 may identify that the subject 102 is experiencing a shockable arrhythmia by analyzing the ECG. Optionally, the secondary defibrillator identifies that the subject 102 is experiencing the shockable arrhythmia by also analyzing an additional physiological parameter, such as blood pressure, blood oxygenation, or the like. Shockable arrhythmias include any arrhythmia that is treatable by administration of an electrotherapy, such as ventricular fibrillation (VF) or ventricular tachycardia (VT) (e.g., pulseless VT) (treatable by defibrillation), atrial fibrillation (AF) (treatable by synchronized cardioversion), or bradycardia (treatable by pacing). For example, the secondary defibrillator 106 may infer that the subject 102 is experiencing pulseless VT by determining that the ECG of the subject 102 is indicative of tachycardia and a plethysmograph of the subject 102 indicates that the heart of the subject 102 is not spontaneously pumping blood through the body of the subject 102.

In some examples, the secondary defibrillator 106 is configured to output, to the bystander 104, a recommendation to administer a treatment to the subject 102. For example, the secondary defibrillator 106 may output audible instructions, visual instructions, or a combination thereof, to administer the treatment. In some examples, the secondary defibrillator 106 instructs the bystander 104 to administer chest compressions to the subject 102. According to some cases, the secondary defibrillator 106 coaches the bystander 104 on administering effective chest compressions. For example, the secondary defibrillator 106 may output an indication of a rate, depth, position, or other treatment parameter associated with optimizing the chest compressions for circulating blood through the body of the subject 102.

In various cases, the secondary defibrillator 106 is configured to output a recommendation to administer an electrical shock to the subject 102. For example, the secondary defibrillator 106 is configured to output audible instructions, visual instructions, or a combination thereof, to press a shock button on the secondary defibrillator 106 that will cause the secondary defibrillator 106 to output an electrical shock to the secondary electrode pads 108. The electrical shock, for example, is a defibrillating electrical shock. For example, the secondary defibrillator 106 outputs the instruction in response to detecting the shockable arrhythmia. Once the bystander 104 presses the shock button, the secondary defibrillator 106 outputs the electrical shock to the heart of the subject along the first shock vector via the secondary electrode pads 108.

In various implementations, the medical condition of the subject 102 persists and/or recurs until a rescuer 110 arrives in the environment 100. In particular cases, the subject 102 has VF that is resistant to attempted defibrillation by the secondary defibrillator 106. For example, the VF of the subject 102 may temporarily resolve in response to the secondary defibrillator 106 outputting the electrical shock to the secondary electrode pads 108. However, the subject 102 may have VF that recurs after the electrical shock is output (e.g., recurrent VF). In some cases, the VF of the subject does not even temporarily abate in response to administration of the electrical shock (e.g., refractory VF). In various cases, the subject 102 has a treatment-resistant shockable arrhythmia.

Upon arriving in the environment 100, the rescuer 110 may utilize a primary defibrillator 112 to monitor and treat the subject 102. In some cases, the rescuer 110 has specialized medical knowledge. For instance, the rescuer 110 may be an emergency medical services (EMS) provider, a physician, a nurse, or some other type of clinical care provider. In various cases, the primary defibrillator 112 is a monitor-defibrillator.

The rescuer 110 applies primary electrode pads 114 to the skin of the subject 102. In various cases, the primary electrode pads 114 are disposed at a second shock vector that is different than the first shock vector. In the example illustrated in FIG. 1, the primary electrode pads 114 are separate from the secondary electrode pads 108. However, in some implementations, the primary electrode pads 114 are physically integrated with the secondary electrode pads 108.

The primary defibrillator 112 includes one or more measurement circuits configured to detect one or more physiological parameters of the subject 102. In various examples, the primary defibrillator 112 is connected to one or more sensors associated with the subject 102. In particular examples, the primary defibrillator 112 is configured to detect the ECG of the subject 102 via electrodes included in the primary electrode pads 114.

The rescuer 110 and/or the primary defibrillator 112, in various cases, determine that the serious medical condition of the subject 102 can be addressed by a multi-shock therapy. As used herein, the term “multi-shock therapy,” and its equivalents, refers to the administration of at least two electrical shocks to a subject in order to treat a shockable arrhythmia of the subject 102. In various cases, at least one of the electrical shocks has an energy level in a range of 150 Joules (J) to 360 J. In some cases, each of the electrical shocks has an energy level in the range of 150 J to 360 J. A delay may exist between leading edges of the electrical shocks. For instance, the delay may be in a range of 0 to 100 milliseconds (ms), such as a delay of 10 ms. In various implementations, the electrical shocks of a multi-shock therapy are temporally overlapping. For example, a leading edge of a secondary electrical shock occurs after the leading edge of a primary electrical shock and before the trailing edge of the primary electrical shock. In an example multi-shock therapy, there is at least one time interval in which the subject receives two defibrillating shocks simultaneously. In various cases, each of the electrical shocks is multiphasic (e.g., biphasic).

In various cases, the electrical shocks may be administered as part of a defibrillation therapy (e.g., the multi-shock therapy is a DSD therapy) or a synchronized cardioversion therapy. In some cases, the multi-shock therapy is a DSD therapy including the administration of multiple electrical shocks to treat VF or VT. In some examples, the multi-shock therapy may be used to treat atrial fibrillation (AF) of the subject 102 by the administration of multiple electrical shocks that are synchronized with characteristics of the ECG of the subject 102. For example, at least one of the electrical shocks may be administered during a QRS complex and/or R-wave and before the T-Wave of the subject 102, which can by identified by an analysis of the ECG of the subject 102. In some examples, the electrical shocks may be pacing pulses that are administered as part of a sequential pacing therapy. For instance, the electrical shocks may be administered sequentially as a treatment for bradycardia exhibited by the subject 102.

Various characteristics of the subject 102 indicate that the multi-shock therapy is warranted. In some cases, the multi-shock therapy is associated with greater risks to the subject 102 than a conventional electrical shock. For example, the multi-shock therapy may be associated with a higher risk of burns than a single-shock therapy. Accordingly, the multi-shock therapy may be indicated in only a subset of types of patient conditions associated with shockable arrhythmias.

In particular cases, the rescuer 110 and/or the primary defibrillator 112 determines that the multi-shock therapy is warranted in response to determining that the subject 102 has a treatment-resistant shockable arrhythmia, such as refractory VF. For example, the bystander 104 and/or the secondary defibrillator 106 may report, to the rescuer 110, that the subject 102 has a treatment-resistant shockable arrhythmia in spite of being administered with an electrotherapy (e.g., one or more previous electrical shocks) from the secondary defibrillator 106. In some cases, the primary defibrillator 112 receives a communication signal from the secondary defibrillator 106 indicating the treatment-resistant shockable arrhythmia. According to various implementations, the rescuer 110 and/or the primary defibrillator 112 may analyze one or more physiological parameters of the subject 102 in order to determine that the multi-shock therapy is warranted.

In various examples, it is preferred to administer the multi-shock therapy using coordinated electrical shocks output by both the primary defibrillator 112 and the secondary defibrillator 106. For example, the primary defibrillator 112 is configured to output an electrical shock by discharging a capacitor. The capacitor, however, may be unable to carry enough charge to enable the primary defibrillator 112 to output multiple defibrillation-level electrical shocks (e.g., whose leading edges are within 10 seconds(s) of each other) by discharging the capacitor. While it may be possible to include multiple capacitors in the primary defibrillator 112, such additional circuitry can greatly increase the weight of the primary defibrillator 112. Thus, limiting the primary defibrillator 112 to a single capacitor for the purpose of defibrillation may enhance the portability of the primary defibrillator 112.

In some cases, it is additionally preferred to output the multiple electrical shocks of a multi-shock therapy at a specific timing relationship. Certain relative timing relationships between the electrical shocks can, in some cases, result in harm or ineffective treatments to the subject 102.

Accordingly, various implementations of the present disclosure relate to coordinating the primary defibrillator 112 and the secondary defibrillator 106 to respectively output electrical shocks in a multi-shock therapy at a predetermined and precise timing relationship. In some cases, the primary defibrillator 112 enters a multi-shock mode. For example, the rescuer manually transitions the primary defibrillator 112 into the multi-shock mode by interacting with one or more input devices of the primary defibrillator 112. In some cases, the primary defibrillator 112 enters the multi-shock mode in response to detecting that the multi-shock therapy is warranted. For instance, the primary defibrillator 112 only enables the rescuer 110 to activate the multi-shock mode in response to determining that the subject 102 has a treatment-resistant shockable arrhythmia. In some cases, the primary defibrillator 112 is configured to enter the multi-shock mode in response to detecting the presence of the secondary defibrillator 106. For example, the primary defibrillator 112 activates the multi-shock mode in response to receiving a communication signal (e.g., a pairing request) from the secondary defibrillator 106.

While in the multi-shock mode, the primary defibrillator 112 is configured to coordinate the administration of the multi-shock therapy with the secondary defibrillator 106. Further, the primary defibrillator 112 is configured to output a primary electrical shock of the multi-shock therapy to the subject 102.

In various cases, the primary defibrillator 112 indicates, to the rescuer 110, that the multi-shock therapy is warranted. In response, the rescuer 110 initiates the multi-shock therapy by interacting with an input device 116 of the primary defibrillator 112. In some cases, the input device 116 is a shock button of the primary defibrillator 112. For example, the primary defibrillator 112 causes the shock button to blink in response to determining that the multi-shock therapy is warranted, and the rescuer 110 presses the shock button in response to viewing the blinking shock button. In some cases, the capacitor of the primary defibrillator 112 is precharged when the rescuer 110 interacts with the input device 116. In some examples, the capacitor of the primary defibrillator 112 is automatically precharged when the treatment-resistant shockable arrhythmia, or any other conditions indicating that the multi-shock therapy is warranted, is detected.

To accommodate the precise timing between the electrical shocks of the multi-shock therapy, the primary defibrillator 112 may refrain from outputting the primary electrical shock immediately after the rescuer 110 interacts with the input device 116. Instead, the primary defibrillator 112 waits for expiration of a delay period that extends from the time at which the input device 116 receives an input signal from the rescuer 110, before outputting the primary electrical shock. The delay period, in some cases, is a predetermined time period. For example, the delay period may have a length in a range of one second to one minute, such as in a range of one second to thirty seconds.

In various cases, the primary defibrillator 112 instructs the secondary defibrillator 106, during the delay period, to output a secondary electrical shock at a time that is optimal with respect to the primary electrical shock. During the delay period, the primary defibrillator 112 transmits a shock instruction 118 to the secondary defibrillator 106. The shock instruction 118, in various cases, causes the secondary defibrillator 106 to output the secondary electrical shock. For instance, the shock instruction 118 includes an indication of a time at which the primary defibrillator 112 will apply the primary electrical shock and/or an indication of an optimal time at which the secondary defibrillator 106 is to apply the secondary electrical shock.

In particular examples, the primary defibrillator 112 utilizes the delay period to support a coordinated application of synchronized cardioversion. In various cases, the primary defibrillator 112 is configured to detect a characteristic of the ECG of the subject, such as a first QRS complex and/or R-wave of the subject. After the delay period and in response to detecting the characteristic, the primary defibrillator 112 outputs the primary electrical shock, which temporally overlaps with or immediately follows the characteristic. In various cases, the shock instruction 118 causes the secondary defibrillator 106 to detect the next instance of the characteristic, and to output the secondary electrical shock in response to detecting the next instance of the characteristic. The delay period, for example, prevents the primary defibrillator 112 and the secondary defibrillator 106 from detecting and applying electrical shocks at the time of the same characteristic of the ECG. For example, the delay period may increase the certainty that the secondary defibrillator 106 will apply the secondary electrical shock after the primary defibrillator 112 outputs the primary electrical shock.

In some cases, the shock instruction 118 causes the secondary defibrillator 106 to activate a multi-shock mode. In various cases, the multi-shock mode is activated in response to the secondary defibrillator 106 receiving another communication signal from the primary defibrillator 112. The multi-shock mode, for the secondary defibrillator 106, may be a protected mode. For example, the secondary defibrillator 106 may deactivate one or more sensors, because the primary defibrillator 112 may operate as the primary monitor of the subject 102. In some cases, the deactivation of the sensor(s) prevent damage to the sensor(s), or circuitry within the secondary defibrillator 106, when the primary defibrillator 112 outputs the primary electrical shock. In some examples, the secondary defibrillator 106 activates a protection circuit within the secondary defibrillator 106 in the multi-shock mode. For example, the secondary defibrillator 106 connects a circuit including one or more diodes that reduce an amount of current flowing through circuitry within the secondary defibrillator 106 caused by the primary electrical shock. In some cases, the secondary defibrillator 106 deactivates one or more output devices in response to entering the multi-shock mode. For example, the secondary defibrillator 106 deactivates a speaker, a display, or some other output device in response to entering the multi-shock mode. Accordingly, the secondary defibrillator 106 may refrain from distracting or confusing the rescuer 110 in the environment 100, when the primary defibrillator 112 is being utilized.

In various implementations, after the expiration of the delay period, the primary defibrillator 112 outputs the primary electrical shock to the primary electrode pads 114 along the second shock vector. Further, the secondary defibrillator 106 outputs the secondary electrical shock to the secondary electrode pads 108 along the first shock vector. In various cases, the multi-shock therapy that includes the primary electrical shock and the secondary electrical shock can treat the serious medical condition of the subject 102. For example, in some examples, the multi-shock therapy permanently resolves the treatment-resistant shockable arrhythmia of the subject 102.

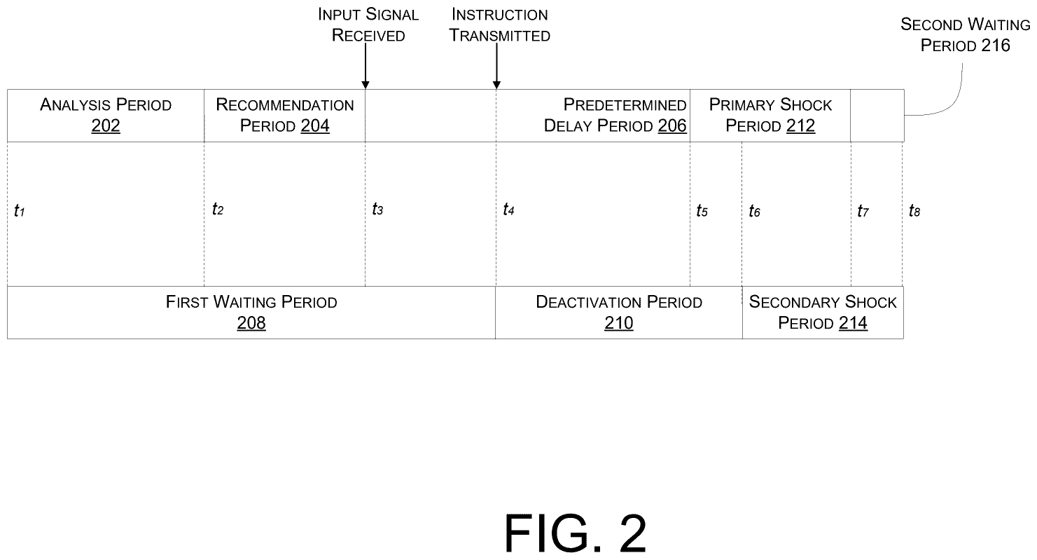

FIG. 2 illustrates an example timing relationship between actions of a primary defibrillator and actions of a secondary defibrillator configured to administer a multi-shock therapy. In FIG. 2, the upper bar represents actions of the primary defibrillator and the lower bar represents actions of the secondary defibrillator. Furthermore, time increases from left to right in FIG. 2. In some examples, the primary defibrillator referenced in FIG. 2 is the primary defibrillator 112 and/or the secondary defibrillator referenced in FIG. 2 is the secondary defibrillator 106.

The primary defibrillator, in various cases, is configured to identify a condition of a subject. During an analysis period 202, the primary defibrillator analyzes one or more physiological parameters of a subject. In some cases, the primary defibrillator detects the physiological parameter(s) during the analysis period 202. For example, the primary defibrillator detects an ECG of the subject and determines that the ECG is indicative of a shockable arrhythmia during the analysis period 202. According to some cases, additional information related to previous events of the subject are additionally analyzed during the analysis period 202. For example, the presence of refractory VF can be identified by determining that VF of the subject failed to even temporarily resolve after the application of a previous electrical shock.

The analysis period 202 extends from a first time t1 to a second time t2. In some implementations, a user (e.g., a rescuer) identifies the shockable arrhythmia during the analysis period 202.

During a recommendation period 204, the primary defibrillator outputs a recommendation to administer a multi-shock therapy. For example, the primary defibrillator outputs the recommendation visually on a display and/or audibly via a speaker. The primary defibrillator outputs the recommendation during the recommendation period 204 in response to determining, by analyzing the physiological parameter(s), that the subject has a medical condition that is treatable by the multi-shock therapy. The recommendation period extends from the second time t2 to the third time t3, for instance.

At the third time t3, the primary defibrillator receives an input signal from a rescuer. The input signal, for instance, causes the primary defibrillator to initiate the multi-shock therapy. For example, the rescuer may press a shock button on the primary defibrillator at the third time t3.

Rather than outputting an electrical shock immediately, the primary defibrillator waits for a predetermined delay period 206 after receiving the input signal. During the predetermined delay period 206, at a fourth time t4, the primary defibrillator transmits an instruction to the secondary defibrillator that coordinates the multi-shock therapy. In various cases, the instruction is transmitted to the secondary defibrillator wirelessly. The predetermined delay period 206 extends from the third time t3 to a fifth time t5.

Meanwhile, prior to receiving the instruction transmitted at the fourth time t4, the secondary defibrillator is in a standby mode. For example, the secondary defibrillator is in a multi-shock mode, in which the secondary defibrillator is configured to wait for instructions from the primary defibrillator before outputting a treatment. The secondary defibrillator is in a first waiting period 208 from the first time t1 to the time at which the instruction is received. Although FIG. 2 illustrates the first waiting period 208 extending to the fourth time t4, it should be noted that in many cases, the first waiting period 208 ends after the fourth time t4, due to a delay in the transmission of the instruction from the primary defibrillator to the secondary defibrillator.

In various cases, the secondary defibrillator enters a deactivation period 210 in response to receiving the instruction from the primary defibrillator. According to some cases, the second defibrillator activates the deactivation period 210 when the primary defibrillator and the secondary defibrillator are detected as being connected to the same subject. In some cases, the secondary defibrillator deactivates one or more sensors during the deactivation period 210. In some examples, the secondary defibrillator activates a protection circuit during the deactivation period 210. In various implementations, the secondary defibrillator deactivates one or more output devices during the deactivation period 210. The deactivation period extends until a sixth time t6.

A primary shock period 212 of the primary defibrillator temporally overlaps with the deactivation period 210 of the secondary defibrillator. During the primary shock period 212, the primary defibrillator outputs a primary electrical shock to the subject. In various cases, the primary electrical shock is externally applied to the skin of the subject. The primary electrical shock is a multiphasic shock, in various cases. For example, the primary electrical shock is a biphasic shock. The primary shock period 212 extends from the fifth time t5 to a seventh time t7.

Based on the instruction from the primary defibrillator, the secondary defibrillator outputs a secondary electrical shock during a secondary shock period 214. In various implementations, the secondary electrical shock is externally applied to the skin of the subject. In various cases, the secondary electrical shock is a multiphasic shock. According to some cases, the secondary electrical shock is administered to the subject at a different shock vector than the primary electrical shock. The secondary shock period 214 partially overlaps the primary shock period 212. For example, the secondary shock period 214 extends from the sixth time t6 (which is before the end of the primary shock period 212 at the seventh time t7) to an eighth time t8. In some alternate implementations, the primary and secondary electrical shocks are simultaneous and/or sequential (e.g., t6=t5 or t6>t7).

In some implementations, the primary defibrillator enters a second waiting period 216 after administration of the primary shock period 212. During the second waiting period 216, the primary defibrillator may avoid damage from the secondary shock by refraining from activating sensors of the primary defibrillator. The second waiting period 216 extends from the seventh time t7 to the eighth time t8. Although specifically illustrated in FIG. 2, in some cases, the primary defibrillator reenters an analysis period after the secondary shock period 214.

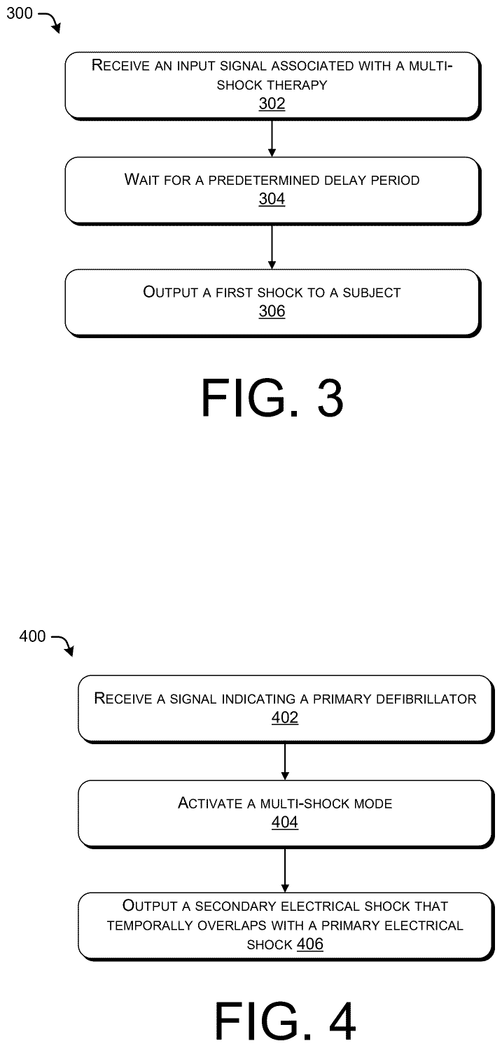

FIG. 3 illustrates an example process 300 for coordinating a multi-shock therapy using a delay period. The process 300 is performed by an entity, which may include a medical device, a defibrillator (e.g., the primary defibrillator 112), at least one processor, or a combination thereof.

At 302, the entity receives an input signal associated with a multi-shock therapy. The multi-shock therapy, for example, includes administrating a first shock and a second shock to a subject. In some cases, the first shock and the second shock are scheduled to be temporally overlapping, at least in part. In some examples, the first shock and the second shock are sequentially applied, such that they are not temporally overlapping. The first shock, for example, is administered via a first vector. For instance, the first shock is administered via electrodes disposed along the first vector. In some examples, the second shock is administered via a second vector. For example, the second shock is administered via electrodes disposed along the second vector. The first vector and the second vector are different vectors, for instance.

In some examples, the entity determines that the subject has a predetermined condition. For example, the entity may determine that an ECG of the subject is indicative of a shockable arrhythmia, such as VF or pulseless VT. In some examples, the entity infers that the subject has a shock-resistant arrhythmia, like refractory VF. For example, the entity may determine that the subject has an ECG indicative of VF or pulseless VT after receiving a previous electrical shock. In some cases, the entity determines that the ECG indicates the subject has refibrillated after administration of the previous electrical shock. For example, a defibrillator may have previously applied, in response to an input signal, a single-shock therapy to the subject while operating in a single-shock mode. The input signal, in some examples, is received in response to determining that the subject has the predetermined condition.

At 304, the entity waits for a predetermined delay period. In some cases, the predetermined delay period is in a range of 10 milliseconds to 1 minute, such as a range of 30 milliseconds to 30 seconds. In some cases, the predetermined delay period is longer than a charging period of the secondary defibrillator. For instance, the charging period can be a time period that the secondary defibrillator utilizes to charge a capacitor from a power source (e.g., a battery), wherein the secondary defibrillator is configured to output the second shock by discharging the capacitor. In some cases, the predetermined delay period is longer than a communication period, in which the instruction is transmitted by the primary defibrillator, received by the secondary defibrillator, and processed by the secondary defibrillator. In some cases, the predetermined delay period is a time in which the secondary defibrillator synchronizes a clock with the primary defibrillator, to enable the precise timing relationship between the two defibrillators. In various cases, the predetermined delay period is longer than a delay period between the input signal and the delivery of the single-shock therapy.

At 306, the entity outputs the first shock to the subject. For instance, the entity outputs the first shock to the electrodes disposed at the first vector.

FIG. 4 illustrates an example process 400 for protecting circuitry associated with a defibrillator during administration of a multi-shock therapy. The process 400 is performed by an entity, which may include a medical device, a defibrillator (e.g., the secondary defibrillator 106), at least one processor, or a combination thereof.

At 402, the entity receives a signal indicating a primary defibrillator. In some implementations, the entity detects the signal via a sensor. The signal, in some cases, indicates a proximity of the primary defibrillator to a secondary defibrillator. In some implementations, the signal includes an instruction to output a secondary electrical shock to a subject. For instance, the instruction indicates a time interval at which the secondary electrical shock is to be delivered. In some cases, the time at which the signal is transmitted (e.g., by the primary defibrillator) and the time interval at which the secondary electrical shock is to be delivered is separated by a delay period.

At 404, the entity activates a multi-shock mode. In some cases, the entity activates the multi-shock mode by deactivating a sensor. For instance, the sensor includes a measurement circuit configured to detect an ECG or transthoracic impedance of the subject. According to some cases, the deactivated sensor is different than the sensor that detected the signal. In some examples, an alarm of the secondary defibrillator is silenced in the multi-shock mode. In some cases, a protection circuit is connected to one or more sensors of the secondary defibrillator when the multi-shock mode is activated.

At 406, the entity outputs a secondary electrical shock that temporally overlaps with a primary electrical shock output by the primary defibrillator. In various cases, a delay period between a leading edge of the primary electrical shock and a leading edge of the secondary electrical shock is in a range of −250 milliseconds (ms) to 250 milliseconds. In some examples, a delay period between a lagging edge of the primary electrical shock and a lagging edge of the secondary electrical shock is in a range of −250 ms to 250 ms. According to some cases, the leading edge of the secondary electrical shock occurs after the leading edge of the primary electrical shock and before the lagging edge of the primary electrical shock. In some examples, the leading edge of the primary electrical shock occurs after the leading edge of the secondary electrical shock and before the lagging edge of the secondary electrical shock. In some cases, one or both of the electrical shocks are multiphasic (e.g., biphasic).

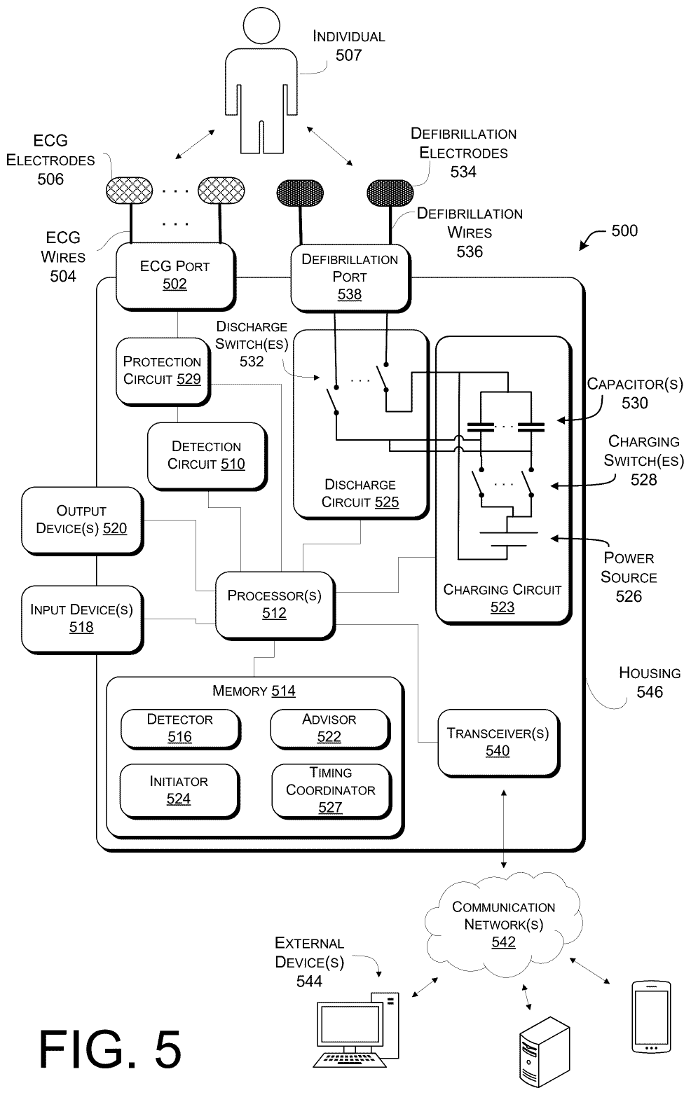

FIG. 5 illustrates an example of an external defibrillator 500 configured to perform various functions described herein. For example, the external defibrillator 500 is the primary defibrillator 112 or secondary defibrillator 106 described above with reference to FIG. 1.

The external defibrillator 500 includes an electrocardiogram (ECG) port 502 connected to multiple ECG wires 504. In some cases, the ECG wires 504 are removeable from the ECG port 502. For instance, the ECG wires 504 are plugged into the ECG port 502 via connectors. The ECG wires 504 are connected to ECG electrodes 506, respectively. In various implementations, the ECG electrodes 506 are disposed on different locations on an individual 508. A detection circuit 510 (also referred to as a “measurement circuit”) is configured to detect relative voltages between the ECG electrodes 506. These voltages are indicative of the electrical activity of the heart of the individual 508.

In various implementations, the ECG electrodes 506 are in contact with the different locations on the skin of the individual 508. In some examples, a first one of the ECG electrodes 506 is placed on the skin between the heart and right arm of the individual 508, a second one of the ECG electrodes 506 is placed on the skin between the heart and left arm of the individual 508, and a third one of the ECG electrodes 506 is placed on the skin between the heart and a leg (either the left leg or the right leg) of the individual 508. In these examples, the detection circuit 510 is configured to measure the relative voltages between the first, second, and third ECG electrodes 506. Respective pairings of the ECG electrodes 506 are referred to as “leads,” and the voltages between the pairs of ECG electrodes 506 are known as “lead voltages.” In some examples, more than three ECG electrodes 506 are included, such that 5-lead or 12-lead ECG signals are detected by the detection circuit 510.

The detection circuit 510 includes at least one analog circuit, at least one digital circuit, or a combination thereof. The detection circuit 510 receives the analog electrical signals from the ECG electrodes 506, via the ECG port 502 and the ECG wires 504. In some cases, the detection circuit 510 includes one or more analog filters configured to filter noise and/or artifact from the electrical signals. The detection circuit 510 includes an analog-to-digital (ADC) in various examples. The detection circuit 510 generates a digital signal indicative of the analog electrical signals from the ECG electrodes 506. This digital signal can be referred to as an “ECG signal” or an “ECG.”

In some cases, the detection circuit 510 further detects an electrical impedance between at least one pair of the ECG electrodes 506. For example, the detection circuit 510 includes, or otherwise controls, a power source that applies a known voltage (or current) across a pair of the ECG electrodes 506 and detects a resultant current (or voltage) between the pair of the ECG electrodes 506. The impedance is generated based on the applied signal (voltage or current) and the resultant signal (current or voltage). In various cases, the impedance corresponds to respiration of the individual 508, chest compressions performed on the individual 508, and other physiological states of the individual 508. In various examples, the detection circuit 510 includes one or more analog filters configured to filter noise and/or artifact from the resultant signal. The detection circuit 510 generates a digital signal indicative of the impedance using an ADC. This digital signal can be referred to as an “impedance signal” or an “impedance.”

The detection circuit 510 provides the ECG signal and/or the impedance signal one or more processors 512 in the external defibrillator 500. In some implementations, the processor(s) 512 includes a central processing unit (CPU), a graphics processing unit (GPU), both CPU and GPU, or other processing unit or component known in the art.

The processor(s) 512 is operably connected to memory 514. In various implementations, the memory 514 is volatile (such as random access memory (RAM)), non-volatile (such as read only memory (ROM), flash memory, etc.) or some combination of the two. The memory 514 stores instructions that, when executed by the processor(s) 512, causes the processor(s) 512 to perform various operations. In various examples, the memory 514 stores methods, threads, processes, applications, objects, modules, any other sort of executable instruction, or a combination thereof. In some cases, the memory 514 stores files, databases, or a combination thereof. In some examples, the memory 514 includes, but is not limited to, RAM, ROM, electrically erasable programmable read-only memory (EEPROM), flash memory, or any other memory technology. In some examples, the memory 514 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 512 and/or the external defibrillator 500. In some cases, the memory 514 at least temporarily stores the ECG signal and/or the impedance signal.

In various examples, the memory 514 includes a detector 516, which causes the processor(s) 512 to determine, based on the ECG signal and/or the impedance signal, whether the individual 508 is exhibiting a particular heart rhythm. For instance, the processor(s) 512 determines whether the individual 508 is experiencing a shockable rhythm that is treatable by defibrillation. Examples of shockable rhythms include VF and ventricular tachycardia (V-Tach). In some examples, the processor(s) 512 determines whether any of a variety of different rhythms (e.g., asystole, sinus rhythm, atrial fibrillation (AF), etc.) are present in the ECG signal.

The processor(s) 512 is operably connected to one or more input devices 518 and one or more output devices 520. Collectively, the input device(s) 518 and the output device(s) 520 function as an interface between a user and the defibrillator 500. The input device(s) 518 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 520 includes at least one of a display, a speaker, a haptic output device, a printer, or any combination thereof. In various examples, the processor(s) 512 causes a display among the input device(s) 518 to visually output a waveform of the ECG signal and/or the impedance signal. In some implementations, the input device(s) 518 includes one or more touch sensors, the output device(s) 520 includes a display screen, and the touch sensor(s) are integrated with the display screen. Thus, in some cases, the external defibrillator 500 includes a touchscreen configured to receive user input signal(s) and visually output physiological parameters, such as the ECG signal and/or the impedance signal.

In some examples, the memory 514 includes an advisor 522, which, when executed by the processor(s) 512, causes the processor(s) 512 to generate advice and/or control the output device(s) 520 to output the advice to a user (e.g., a rescuer). In some examples, the processor(s) 512 provides, or causes the output device(s) 520 to provide, an instruction to perform CPR on the individual 508. In some cases, the processor(s) 512 evaluates, based on the ECG signal, the impedance signal, or other physiological parameters, CPR being performed on the individual 508 and causes the output device(s) 520 to provide feedback about the CPR in the instruction. According to some examples, the processor(s) 512, upon identifying that a shockable rhythm is present in the ECG signal, causes the output device(s) 520 to output an instruction and/or recommendation to administer a defibrillation shock to the individual 508.

The memory 514 also includes an initiator 524 which, when executed by the processor(s) 512, causes the processor(s) 512 to control other elements of the external defibrillator 500 in order to administer a defibrillation shock to the individual 508. In some examples, the processor(s) 512 executing the initiator 524 selectively causes the administration of the defibrillation shock based on determining that the individual 508 is exhibiting the shockable rhythm and/or based on an input from a user (received, e.g., by the input device(s) 518. In some cases, the processor(s) 512 causes the defibrillation shock to be output at a particular time, which is determined by the processor(s) 512 based on the ECG signal and/or the impedance signal.

In various cases, the memory 514 further includes a timing coordinator 527 that, when executed by the processor(s) 512, causes the processor(s) 512 to activate a multi-shock mode of the defibrillator 500 and/or coordinate timing of electrical shocks in a multi-shock therapy with a secondary defibrillator. For example, the processor(s) 512 generate a shock instruction that causes the secondary defibrillator to output a secondary electrical shock at a future time. The defibrillator 500, in various cases, is configured to transmit the shock instruction during a predetermined delay period. After expiration of the predetermined delay period, the defibrillator 500 may output a primary electrical shock during a time period that at least partially overlaps with the administration of the secondary electrical shock.

In some implementations, the processor(s) 512 selectively activate a protection circuit 529 in response to executing at least some instructions in the timing coordinator 527. For example, the processor(s) 512 connect the protection circuit 259 to a path between the ECG port 502 and the detection circuit 510 in response to activating the multi-shock mode. The protection circuit 259, for instance, includes one or more diodes that prevent current from being induced in the detection circuit 510 due to administration of an electrical shock to the individual 507 by another defibrillator coupled with the individual 507. In some cases, the processor(s) 512 deactivates the input device(s) 518 and/or the output device(s) 520 when the defibrillator 500 is in the multi-shock mode.

The processor(s) 512 is operably connected to a charging circuit 523 and a discharge circuit 525. In various implementations, the charging circuit 523 includes a power source 526, one or more charging switches 528, and one or more capacitors 530. The power source 526 includes, for instance, a battery. The processor(s) 512 initiates a defibrillation shock by causing the power source 526 to charge at least one capacitor among the capacitor(s) 530. For example, the processor(s) 512 activates at least one of the charging switch(es) 528 in the charging circuit 523 to complete a first circuit connecting the power source 526 and the capacitor to be charged. Then, the processor(s) 512 causes the discharge circuit 525 to discharge energy stored in the charged capacitor across a pair of defibrillation electrodes 534, which are in contact with the individual 508. For example, the processor(s) 512 deactivates the charging switch(es) 528 completing the first circuit between the capacitor(s) 530 and the power source 526, and activates one or more discharge switches 532 completing a second circuit connecting the charged capacitor 530 and at least a portion of the individual 508 disposed between defibrillation electrodes 534.

The energy is discharged from the defibrillation electrodes 534 in the form of a defibrillation shock. For example, the defibrillation electrodes 534 are connected to the skin of the individual 508 and located at positions on different sides of the heart of the individual 508, such that the defibrillation shock is applied across the heart of the individual 508. The defibrillation shock, in various examples, depolarizes a significant number of heart cells in a short amount of time. The defibrillation shock, for example, interrupts the propagation of the shockable rhythm (e.g., VF or pulseless VT) through the heart. In some examples, the defibrillation shock is 200 J or greater with a duration of about 0.015 seconds. In some cases, the defibrillation shock has a multiphasic (e.g., biphasic) waveform. The discharge switch(es) 532 are controlled by the processor(s) 512, for example. In various implementations, the defibrillation electrodes 534 are connected to defibrillation leads 536. The defibrillation wires 536 are connected to a defibrillation port 538, in implementations. According to various examples, the defibrillation wires 536 are removable from the defibrillation port 538. For example, the defibrillation wires 536 are plugged into the defibrillation port 538.

In various implementations, the processor(s) 512 is operably connected to one or more transceivers 540 that transmit and/or receive data over one or more communication networks 542. For example, the transceiver(s) 540 includes a network interface card (NIC), a network adapter, a local area network (LAN) adapter, or a physical, virtual, or logical address to connect to the various external devices and/or systems. In various examples, the transceiver(s) 540 includes any sort of wireless transceivers capable of engaging in wireless communication (e.g., radio frequency (RF) communication). For example, the communication network(s) 542 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 540 includes other wireless modems, such as a modem for engaging in WI-FI®, WIGIG®, WIMAX®, BLUETOOTH®, or infrared communication over the communication network(s) 542.

The defibrillator 500 is configured to transmit and/or receive data (e.g., ECG data, impedance data, data indicative of one or more detected heart rhythms of the individual 508, data indicative of one or more defibrillation shocks administered to the individual 508, etc.) with one or more external devices 544 via the communication network(s) 542. The external devices 544 include, for instance, mobile devices (e.g., mobile phones, smart watches, etc.), Internet of Things (IoT) devices, medical devices (e.g., a primary defibrillator or secondary defibrillator), computers (e.g., laptop devices, servers, etc.), or any other type of computing device configured to communicate over the communication network(s) 542. In some examples, the external device(s) 544 is located remotely from the defibrillator 500, such as at a remote clinical environment (e.g., a hospital). According to various implementations, the processor(s) 512 causes the transceiver(s) 540 to transmit data to the external device(s) 544. In some cases, the transceiver(s) 540 receives data from the external device(s) 544 and the transceiver(s) 540 provide the received data to the processor(s) 512 for further analysis. In some cases, the defibrillator 500 is configure to transmit or receive a shock instruction via the communication network(s) 542.

In various implementations, the external defibrillator 500 also includes a housing 546 that at least partially encloses other elements of the external defibrillator 500. For example, the housing 546 encloses the detection circuit 510, the processor(s) 512, the memory 514, the charging circuit 523, the transceiver(s) 540, or any combination thereof. In some cases, the input device(s) 518 and output device(s) 520 extend from an interior space at least partially surrounded by the housing 546 through a wall of the housing 546. In various examples, the housing 546 acts as a barrier to moisture, electrical interference, and/or dust, thereby protecting various components in the external defibrillator 500 from damage.

In some implementations, the external defibrillator 500 is an automated external defibrillator (AED) operated by an untrained user (e.g., a bystander, layperson, etc.) and can be operated in an automatic mode. In automatic mode, the processor(s) 512 automatically identifies a rhythm in the ECG signal, makes a decision whether to administer a defibrillation shock, charges the capacitor(s) 530, discharges the capacitor(s) 530, or any combination thereof. In some cases, the processor(s) 512 controls the output device(s) 520 to output (e.g., display) a simplified user interface to the untrained user. For example, the processor(s) 512 refrains from causing the output device(s) 520 to display a waveform of the ECG signal and/or the impedance signal to the untrained user, in order to simplify operation of the external defibrillator 500.

In some examples, the external defibrillator 500 is a monitor-defibrillator utilized by a trained user (e.g., a clinician, an emergency responder, etc.) and can be operated in a manual mode or the automatic mode. When the external defibrillator 500 operates in manual mode, the processor(s) 512 cause the output device(s) 520 to display a variety of information that may be relevant to the trained user, such as waveforms indicating the ECG data and/or impedance data, notifications about detected heart rhythms, and the like.

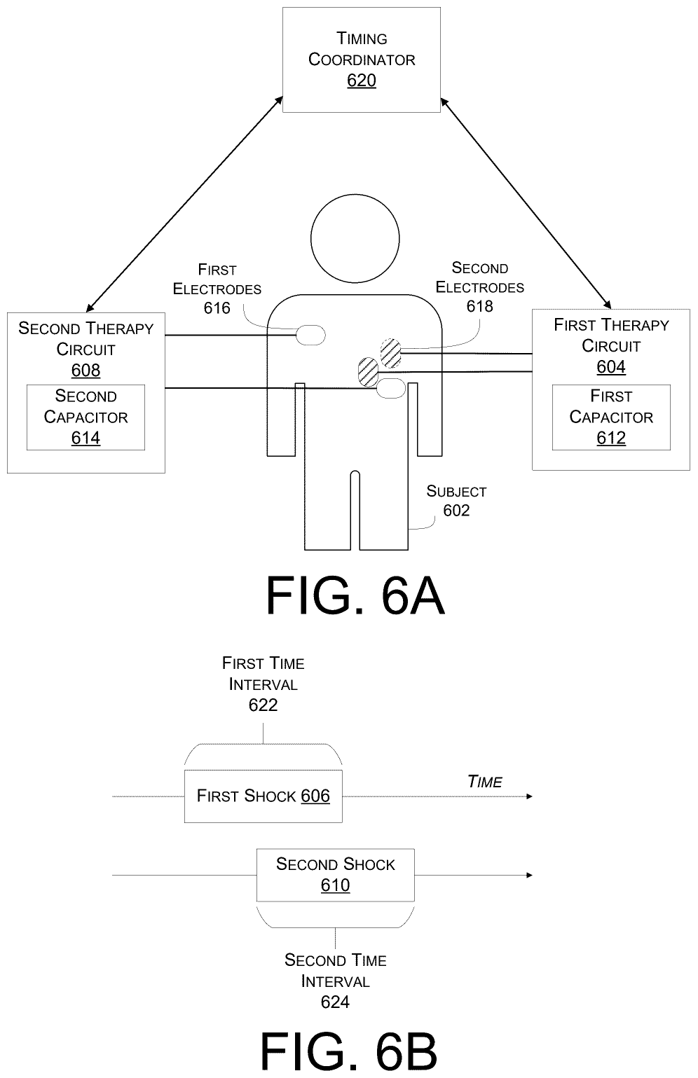

FIGS. 6A and 6B illustrate examples of environments and timing related to administering a multi-shock therapy. FIG. 6A shows an environment configured to administer the multi-shock therapy. FIG. 6B shows a timing relationship of multiple shocks administered in the multi-shock therapy.

In various cases, a subject 602 has a medical condition that is treatable by defibrillation. For example, the subject 602 may have a shockable cardiac arrhythmia, such as VF or pulseless VT. In some cases, however, conventional defibrillation therapies do not resolve the medical condition. For example, the subject 602 may be experiencing VF that does not resolve after the administration of a single biphasic electrical shock administered by a defibrillator. For instance, the subject 602 may have refractory VF.

In various implementations of the present disclosure, the medical condition of the subject 602 is treatable by administration of a multi-shock therapy. Specifically, a first therapy circuit 604 is configured to output a first shock 606 to the subject 602 and a second therapy circuit 608 is configured to output a second shock 610 to the subject 602. In various cases, the first shock 606 and the second shock 610 temporally overlap in time, at least partially. For example, a start time of the second shock 610 occurs after the start time of the first shock 606, but the start time of the second shock 610 occurs before the end time of the first shock 606. In some cases, the second shock 610 is administered after the first shock 606. For instance, a start time of the second shock 610 is simultaneous with, or occurs after, the end time of the first shock 606. In various cases, the first shock 606 is a biphasic shock and/or the second shock 610 is a biphasic shock. In some implementations, the first shock 606 is a monophasic shock and/or the second shock 610 is a biphasic shock. In some cases, the first shock 606 has a shorter duration and/or lower voltage amplitude than the second shock 610.

The first therapy circuit 604 outputs the first shock 606 by discharging a first capacitor 612. Similarly, the second therapy circuit 608 outputs the second shock 610 by discharging a second capacitor 614. In various implementations, one or more power sources are configured to charge the first capacitor 612 and/or the second capacitor 614 prior to discharge. In various cases, the first therapy circuit 604 includes a first H-bridge circuit including the first capacitor 612 and/or the second therapy circuit 608 includes a second H-bridge circuit including the second capacitor 614. The first H-bridge circuit and the second H-bridge circuit are configured to output the first shock 606 and the second shock 610 as biphasic shocks, for instance, via sequential activation of switches in the first H-bridge circuit and the second H-bridge circuit.

The first therapy circuit 604 is configured to output the first shock 606 to first electrodes 616. The second therapy circuit 608 is configured to output the second shock 610 to second electrodes 618. In various cases, the first electrodes 616 and/or the second electrodes 618 are disposed externally on the skin of the subject 602. For example, the first electrodes 616 and/or the second electrodes 618 are adhered to the skin of the subject 602. In various implementations, the first electrodes 616 and the second electrodes 618 are associated with different shock vectors. For instance, a first shock vector extends between the first electrodes 616 and a second shock vector extends between the second electrodes 618, wherein the first shock vector and the second shock vector are different. For example, the first shock vector may be an anterior-lateral position and the second shock vector may be an anterior-posterior position. In various implementations, the first shock vector and the second shock vector both extend through the heart of the subject 602. Although FIG. 6A illustrates the first electrodes 616 as being separate from the second electrodes 618, implementations are not so limited. For example, one electrode may be shared among the first electrodes 616 and the second electrodes 618.

The first therapy circuit 604 and the second therapy circuit 608 are distributed among one or more devices. In some cases, the first therapy circuit 604 is part of a first external defibrillator and the second therapy circuit 608 is part of a second external defibrillator. For example, the first external defibrillator and the second external defibrillator are both monitor-defibrillators, both AEDs, or a monitor-defibrillator and an AED. In some cases, the first therapy circuit 604 or the second therapy circuit 608 is integrated into an accessory device without monitoring capabilities, and which is solely designed to output electrical shocks upon receiving an input signal from a separate device. For example, the accessory device may lack, or be disconnected from, one or more sensors configured to identify one or more physiological parameters of the subject 602. In some cases, the accessory device lacks a display, speaker, or other user interface device. In some implementations, the first therapy circuit 604 and the second therapy circuit 608 are integrated into the same device, such as the same monitor-defibrillator.

Optionally, a timing coordinator 620 is configured to cause the first therapy circuit 604 to output the first shock 606 during a first time interval 622 and/or to cause the second therapy circuit 608 to output the second shock 610 during the second time interval 624. For example, the timing coordinator 620 outputs one or more signals (e.g., electrical signals, communication signals, etc.) to the first therapy circuit 604 and/or the second therapy circuit 608. Upon receiving the signal(s) from the timing coordinator 620, the first therapy circuit 604 may discharge the first capacitor 612 during the first time interval 622 and/or the second therapy circuit 608 may discharge the second capacitor 614 during the second time interval 624. The timing coordinator 620 can be implemented in hardware (e.g., a circuit), software (e.g., instructions executed by at least one processor), or a combination thereof. In some cases, the timing coordinator 620 is a standalone device. Examples of standalone timing devices that can serve as the timing coordinator 620 are described in U.S. Pat. No. 10,981,014, which is incorporated by reference herein in its entirety. In some examples, the timing coordinator 620 is integrated into the same device as the first therapy circuit 604 and/or the second therapy circuit 608.

Various timing relationships between the first time interval 622 and the second time interval 624 can be implemented according to various implementations of the present disclosure. In some cases, a delay between the start times (i.e., the leading edges) of the first time interval 622 and the second time interval 624 is in a range of 0 and 250 milliseconds (ms). In some cases, the delay between the start times of the first time interval 622 and the second time interval 624 is in a range of −250 and 0 ms. Although FIG. 6B illustrates the first time interval 622 and the second time interval 624 as having equivalent durations, implementations are not so limited. For example, the first time interval 622 may be longer or shorter than the second time interval 624. Various timing relationships are described in U.S. Pat. No. 10,702,701, which is incorporated by reference herein in its entirety.

In various cases, the timing coordinator 620 is configured to detect the first shock 606 and may cause the second therapy circuit 608 to output the second shock 610 in response. For example, the timing coordinator 620 may detect a signal indicative of the discharge of the first shock 606, and may output a signal that causes the second therapy circuit 608 to discharge the second shock 610. In some examples, the timing coordinator 620 is inductively coupled with the first therapy circuit 604 and/or the first electrodes 616, which enables the timing coordinator 620 to detect the discharge of the first shock 606. Various techniques for detecting the discharge of a first shock in order to cause the application of a second shock in multi-shock therapy are described in U.S. Pat. Nos. 10,625,088 and 10,632,320, which are incorporated by reference herein in their entirety.

### EXAMPLE CLAUSES

1. A first defibrillator including: a measurement circuit configured to detect an electrocardiogram (ECG) of a subject; a treatment circuit configured to output a primary electrical shock to the subject and to output a secondary electrical shock to the subject; an input device configured to receive an input signal from a rescuer; and a processor configured to: determine, by analyzing the ECG, that the subject has refibrillated after administration of the primary electrical shock; in response to determining that the subject has refibrillated after administration of the primary electrical shock, switch an operating mode of the first defibrillator from a single-shock mode to a multi-shock mode; in response to switching the operating mode of the first defibrillator from the single-shock mode to the multi-shock mode: during a predetermined delay period after the input device has received the input signal from the rescuer, output, to a second defibrillator, an instruction to administer a third electrical shock to the subject, the third electrical shock being temporally overlapping with the secondary electrical shock; and in response to expiration of the predetermined delay period after the input device has received the input signal from the rescuer, cause the treatment circuit to output the secondary electrical shock to the subject.
2. The first defibrillator of clause 1, wherein the treatment circuit is configured to output the secondary electrical shock to first electrodes configured to be disposed along a first vector, and wherein the second defibrillator is configured to output the third electrical shock to second electrodes configured to be disposed along a second vector.
3. The first defibrillator of clause 1 or 2, the input signal being a first input signal, wherein the input device is further configured to receive a second input signal from the rescuer, and wherein the processor is configured to switch the operating mode of the first defibrillator from the single-shock mode to the multi-shock mode further in response to the input device receiving the second input signal.
4. A method, including: receiving, by a defibrillator operating in a multi-shock mode, an input signal associated with a multi-shock therapy, the multi-shock therapy including a first shock administered to a subject and a second shock administered to the subject, the first shock and the second shock being temporally overlapping or sequential; and in response to expiration of a predetermined delay period after receiving the input signal, outputting, by the defibrillator operating in the multi-shock mode, the first shock to the subject.
5. The method of clause 4, wherein the predetermined delay period is in a range of about 30 milliseconds to about 30 seconds.
6. The method of clause 4 or 5, wherein the first shock is associated with a first vector and the second shock is associated with a second vector.
7. The method of any of clauses 4 to 6, further including: determining that an electrocardiogram (ECG) of the subject is indicative of a shockable arrhythmia including ventricular fibrillation (VF) or pulseless ventricular tachycardia (VT), wherein receiving the input signal associated with the multi-shock therapy is in response to determining that the ECG of the subject is indicative of the shockable arrhythmia.
8. The method of clause 7, wherein determining that the ECG of the subject is indicative of the shockable arrhythmia includes: determining that the ECG is indicative of the shockable arrhythmia after administration of an initial shock to the subject; and in response to determining that the ECG exhibits the shockable arrhythmia after administration of the initial shock to the subject, transitioning, by the defibrillator, from a single-shock mode to the multi-shock mode.
9. The method of any of clauses 4 to 8, further including: during the predetermined delay period, outputting a signal to an external device, thereby causing the external device to administer the second shock to the subject.
10. The method of any of clauses 4 to 9, further including: in response to the expiration of the predetermined delay period after receiving the input signal, outputting, by the defibrillator operating in the multi-shock mode, the second shock to the subject.
11. The method of any of clauses 4 to 10, the input signal being a first input signal, the method further including: receiving, by the defibrillator operating in a single-shock mode, a second input signal associated with a single-shock therapy, the single-shock therapy including a third electrical shock administered to the subject; and in response to receiving the second input signal, outputting, by the defibrillator operating in the single-shock mode, the third shock to the subject.
12. The method of clause 11, wherein the predetermined delay period has a greater length than a time period between receiving the second input signal and outputting the third electrical shock to the subject.
13. A defibrillator, including: an input device configured to receive an input signal associated with a multi-shock therapy, the multi-shock therapy including a first shock and a second shock that are temporally overlapping; a treatment circuit configured to output, in response to expiration of a predetermined delay period after receiving the input signal, the first shock of the multi-shock therapy.
14. The defibrillator of clause 13, wherein the predetermined delay period is in a range of about 30 milliseconds to about 30 seconds.
15. The defibrillator of clause 13 or 14, wherein the first shock is associated with a first vector and the second shock is associated with a second vector.
16. The defibrillator of any of clauses 13 to 15, further including: a processor configured to: determine that an electrocardiogram (ECG) of a subject is indicative of a shockable arrhythmia including ventricular fibrillation (VF) or pulseless ventricular tachycardia (VT), wherein receiving the input signal associated with the multi-shock therapy is in response to the processor determining that the ECG of the subject is indicative of the shockable arrhythmia.
17. The defibrillator of clause 16, wherein the processor is configured to determine that the ECG of the subject is indicative of the shockable arrhythmia by: determining that the ECG is indicative of the shockable arrhythmia after administration of an initial shock to the subject; and in response to determining that the ECG exhibits the shockable arrhythmia after administration of the initial shock to the subject, transitioning the defibrillator from a single-shock mode to a multi-shock mode.
18. The defibrillator of any of clauses 13 to 17, further including: a transceiver configured to output, during the predetermined delay period, a signal to an external device, thereby causing the external device to administer the second shock to a subject.
19. The defibrillator of any of clauses 13 to 18, the treatment circuit being a first treatment circuit including a first capacitor, the defibrillator further including: a second treatment circuit including a second capacitor configured to output, in response to the expiration of the predetermined delay period after receiving the input signal, the second shock.
20. The defibrillator of any of clauses 13 to 19, the input signal being a first input signal, wherein the input device is further configured to receive a second input signal associated with a single-shock therapy, the single-shock therapy including a third shock administered, and wherein the treatment circuit is further configured to output, in response to receiving the second input signal, the third shock, and wherein the predetermined delay period has a greater length than a time period between receiving the second input signal and outputting the third shock.
21. A system, including: a primary defibrillator including: a primary measurement circuit configured to detect an electrocardiogram (ECG) of a subject; a primary treatment circuit configured to output a primary electrical shock to the subject; a primary transceiver configured to transmit a multi-shock instruction indicating a secondary time interval; and a primary processor configured to: determine that the ECG of the subject is indicative of a shockable arrhythmia including ventricular fibrillation (VF) or pulseless ventricular tachycardia (VT); and in response to determining that the ECG of the subject is indicative of the shockable arrhythmia: cause the primary transceiver to transmit the multi-shock instruction; and cause the primary treatment circuit to output the primary electrical shock to the subject during a primary time interval; a secondary defibrillator including: a secondary measurement circuit configured to detect the ECG of the subject; a secondary treatment circuit configured to output a secondary electrical shock to the subject; a secondary transceiver configured to receive the multi-shock instruction; and a secondary processor configured to: in response to the secondary transceiver receiving the multi-shock instruction, activate a multi-shock mode by deactivating the secondary measurement circuit; and in response to activating the multi-shock mode, cause the secondary treatment circuit to output the secondary electrical shock to the subject during the secondary time interval.
22. The system of clause 21, wherein the primary defibrillator further includes: an input device configured to receive an input signal from a user at an input time, wherein the primary processor is configured to cause the primary transceiver to transmit the multi-shock instruction further in response to the input device receiving the input signal from the user, and wherein a delay period is between the input time and the primary time interval.
23. The system of clause 21 or 22, wherein the secondary processor is configured to deactivate the secondary measurement circuit by connecting a protection circuit to the secondary measurement circuit.
24. A secondary defibrillator, including: a treatment circuit configured to be coupled with electrodes disposed on skin of a subject; a sensor configured to detect a physiological parameter of the subject; an input device configured to receive a signal indicating a primary defibrillator; and a processor configured to: in response to the input device receiving the signal indicating the primary defibrillator, activating a multi-shock mode by deactivating the sensor; and in response to activating the multi-shock mode: cause the treatment circuit to output a secondary electrical shock, the secondary electrical shock temporally overlapping with a primary electrical shock output by the primary defibrillator.
25. The secondary defibrillator of clause 24, wherein the physiological parameter includes an electrocardiogram (ECG) or a transthoracic impedance of the subject.
26. The secondary defibrillator of clause 24 or 25, wherein the input device includes a transceiver.
27. The secondary defibrillator of any of clauses 24 to 26, the sensor being a first sensor, wherein the input device includes a second sensor.
28. The secondary defibrillator of any of clauses 24 to 27, wherein the signal indicating the primary defibrillator indicates a proximity of the primary defibrillator.
29. The secondary defibrillator of any of clauses 24 to 28, wherein the signal indicating the primary defibrillator further includes an instruction to output the secondary electrical shock at a time interval.
30. The secondary defibrillator of clause 29, wherein the time interval and a time at which the input device receives the signal indicating the primary defibrillator are separated by a delay period.
31. The secondary defibrillator of any of clauses 24 to 30, further including: a display configured to visually present an indication of the physiological parameter of the subject; and a speaker configured to audibly output an alarm, wherein the processor is configured to deactivate the sensor by: causing the display to refrain from visually presenting the indication of the physiological parameter of the subject; and causing the speaker to silence the alarm.
32. The secondary defibrillator of any of clauses 24 to 31, further including: a protection circuit, wherein the processor is configured to activate the multi-shock mode further by connecting the protection circuit to an electrical path between the sensor and the skin of the subject.
33. A method, including: receiving a signal indicating a primary defibrillator; in response to receiving the signal indicating the primary defibrillator, activating a multi-shock mode by deactivating a sensor of a secondary defibrillator; and in response to activating the multi-shock mode, outputting a secondary electrical shock that temporally overlaps with a primary electrical shock output by the primary defibrillator.
34. The method of clause 33, wherein sensor is configured to detect an ECG or a transthoracic impedance of a subject.
35. The method of clause 33 or 34, the sensor being a first sensor, wherein receiving the signal indicating the primary defibrillator includes detecting, by a second sensor, the signal.
36. The method of any of clauses 33 to 35, wherein the signal indicating the primary defibrillator indicates a proximity of the primary defibrillator to the secondary defibrillator.
37. The method of any of clauses 33 to 36, wherein the signal indicating the primary defibrillator further includes an instruction to output the secondary electrical shock at a time interval.
38. The method of clause 37, wherein the time interval and a time at which the signal indicating the primary defibrillator is received are separated by a delay period.
39. The method of any of clauses 33 to 38, wherein deactivating the sensor includes: silencing an alarm of the secondary defibrillator.
40. The method of any of clauses 33 to 39, wherein activating the multi-shock mode includes connecting a protection circuit to an electrical path between the sensor and skin of a subject.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on”is equivalent to “based at least partly on,”unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A first defibrillator comprising:
a measurement circuit configured to detect an electrocardiogram (ECG) of a subject;
a treatment circuit configured to output a primary electrical shock to the subject and to output a secondary electrical shock to the subject;
an input device configured to receive an input signal from a rescuer; and
a processor configured to:
determine, by analyzing the ECG, that the subject has refibrillated after administration of the primary electrical shock;
in response to determining that the subject has refibrillated after administration of the primary electrical shock, switch an operating mode of the first defibrillator from a single-shock mode to a multi-shock mode;
in response to switching the operating mode of the first defibrillator from the single-shock mode to the multi-shock mode:
during a predetermined delay period after the input device has received the input signal from the rescuer, output, to a second defibrillator, an instruction to administer a third electrical shock to the subject, the third electrical shock being temporally overlapping with the secondary electrical shock; and
in response to expiration of the predetermined delay period after the input device has received the input signal from the rescuer, cause the treatment circuit to output the secondary electrical shock to the subject.

2. The first defibrillator of claim 1, wherein the treatment circuit is configured to output the secondary electrical shock to first electrodes configured to be disposed along a first vector, and
wherein the second defibrillator is configured to output the third electrical shock to second electrodes configured to be disposed along a second vector.

3. The first defibrillator of claim 1, the input signal being a first input signal, wherein the input device is further configured to receive a second input signal from the rescuer, and
wherein the processor is configured to switch the operating mode of the first defibrillator from the single-shock mode to the multi-shock mode further in response to the input device receiving the second input signal.

4. A method, comprising:
receiving, by a defibrillator operating in a multi-shock mode, an input signal associated with a multi-shock therapy, the multi-shock therapy comprising a first shock administered to a subject and a second shock administered to the subject, the first shock and the second shock being temporally overlapping or sequential; and
in response to expiration of a predetermined delay period after receiving the input signal, outputting, by the defibrillator operating in the multi-shock mode, the first shock to the subject.

5. The method of claim 4, wherein the predetermined delay period is in a range of about 30 milliseconds to about 30 seconds.

6. The method of claim 4, wherein the first shock is associated with a first vector and the second shock is associated with a second vector.

7. The method of claim 4, further comprising:
determining that an electrocardiogram (ECG) of the subject is indicative of a shockable arrhythmia comprising ventricular fibrillation (VF) or pulseless ventricular tachycardia (VT),
wherein receiving the input signal associated with the multi-shock therapy is in response to determining that the ECG of the subject is indicative of the shockable arrhythmia.

8. The method of claim 7, wherein determining that the ECG of the subject is indicative of the shockable arrhythmia comprises:
determining that the ECG is indicative of the shockable arrhythmia after administration of an initial shock to the subject; and
in response to determining that the ECG exhibits the shockable arrhythmia after administration of the initial shock to the subject, transitioning, by the defibrillator, from a single-shock mode to the multi-shock mode.

9. The method of claim 4, further comprising:
during the predetermined delay period, outputting a signal to an external device, thereby causing the external device to administer the second shock to the subject.

10. The method of claim 4, further comprising:
in response to the expiration of the predetermined delay period after receiving the input signal, outputting, by the defibrillator operating in the multi-shock mode, the second shock to the subject.

11. The method of claim 4, the input signal being a first input signal, the method further comprising:
receiving, by the defibrillator operating in a single-shock mode, a second input signal associated with a single-shock therapy, the single-shock therapy comprising a third electrical shock administered to the subject; and
in response to receiving the second input signal, outputting, by the defibrillator operating in the single-shock mode, the third shock to the subject.

12. The method of claim 11, wherein the predetermined delay period has a greater length than a time period between receiving the second input signal and outputting the third electrical shock to the subject.

13. A defibrillator, comprising:
an input device configured to receive an input signal associated with a multi-shock therapy, the multi-shock therapy comprising a first shock and a second shock that are temporally overlapping;
a treatment circuit configured to output, in response to expiration of a predetermined delay period after receiving the input signal, the first shock of the multi-shock therapy.

14. The defibrillator of claim 13, wherein the predetermined delay period is in a range of about 30 milliseconds to about 30 seconds.

15. The defibrillator of claim 13, wherein the first shock is associated with a first vector and the second shock is associated with a second vector.

16. The defibrillator of claim 13, further comprising:
a processor configured to:
determine that an electrocardiogram (ECG) of a subject is indicative of a shockable arrhythmia comprising ventricular fibrillation (VF) or pulseless ventricular tachycardia (VT),
wherein receiving the input signal associated with the multi-shock therapy is in response to the processor determining that the ECG of the subject is indicative of the shockable arrhythmia.

17. The defibrillator of claim 16, wherein the processor is configured to determine that the ECG of the subject is indicative of the shockable arrhythmia by:
determining that the ECG is indicative of the shockable arrhythmia after administration of an initial shock to the subject; and
in response to determining that the ECG exhibits the shockable arrhythmia after administration of the initial shock to the subject, transitioning the defibrillator from a single-shock mode to a multi-shock mode.

18. The defibrillator of claim 13, further comprising:
a transceiver configured to output, during the predetermined delay period, a signal to an external device, thereby causing the external device to administer the second shock to a subject.

19. The defibrillator of claim 13, the treatment circuit being a first treatment circuit comprising a first capacitor, the defibrillator further comprising:
a second treatment circuit comprising a second capacitor configured to output, in response to the expiration of the predetermined delay period after receiving the input signal, the second shock.

20. The defibrillator of claim 13, the input signal being a first input signal, wherein the input device is further configured to receive a second input signal associated with a single-shock therapy, the single-shock therapy comprising a third shock administered, and
wherein the treatment circuit is further configured to output, in response to receiving the second input signal, the third shock, and
wherein the predetermined delay period has a greater length than a time period between receiving the second input signal and outputting the third shock.
